(19) United States
(12) Patent Application Publication
(10) Pub. No.: US 2008/0206368 A1
(43) Pub. Date: Aug. 28, 2008

(54) Administration of Morinda Citrifolia L. Based Formulations to Increase Birth Rates

(76) Inventor: Mian-Ying Wang, Rockford, IL (US)

Correspondence Address: KIRTON AND MCCONKIE, 60 EAST SOUTH TEMPLE,, SUITE 1800, SALT LAKE CITY, UT, 84111, US

(21) Appl. No.: 12/035,242

(22) Filed: Feb. 21, 2008

Related U.S. Application Data
(60) Provisional application No. 60/891,665, filed on Feb. 26, 2007.

(51) Int. Cl. A61K 36/746; A61K 36/87; A61K 36/45; A61K 36/73; A61P 15/00

(52) U.S. Cl. 424/732; 424/777; 424/766; 424/765

(57) ABSTRACT

The present invention relates to methods and formulations directed to increasing mammalian birth rates comprising the administration of processed Morinda citrifolia based formulations.

### RELATED APPLICATIONS

This application claims priority to U.S. Provisional Patent Application Ser. No. 60/891,665 filed Feb. 26, 2007.

### BACKGROUND

1. Field of Invention

The field of the invention relates to products which may be administered to produce desirable physiological improvement. In particular, the invention relates to the administration of products enhanced with Morinda citrifolia in order to decrease ameliorate infertility.

2. Background

Infertility is the inability to naturally conceive a child or to carry a pregnancy to full term. There are many reasons why a couple may not be able to conceive, or may not be able to conceive without medical assistance.

Infertility affects approximately 10% of people of reproductive age 15% of couples. Roughly 40% of cases involve a male contribution or factor, 40% involve a female factor, and the remainder involves both sexes. There are some health insurance companies that cover diagnosis of infertility but frequently once diagnosed will not cover any treatment costs.

Because most of the common medical treatments for infertility are expensive and can involve serious side effects, compositions containing natural products that would treat infertility are highly desirable.

### SUMMARY OF THE INVENTION

Some embodiments relate to formulations for increasing mammalian birth rates comprising processed Morinda citrifolia products and methods for administering such.

Some embodiments provide a method of treating various diseases and ailments, which comprise administering to said mammal a processed Morinda citrifolia product selected from a group consisting of: extract from the leaves of Morinda citrifolia, leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, Morinda citrifolia seeds, Morinda citrifolia seed extracts, extracts from defatted Morinda citrifolia seeds and evaporated concentration of Morinda citrifolia fruit juice.

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. The scope of the invention is, therefore, indicated by the appended claims rather than by the foregoing description. All changes that come within the meaning and range of equivalency of the claims are to be embraced within their scope.

Embodiments of the present invention feature methods and compositions for increasing mammalian birth rates comprising processed Morinda citrifolia products. The increase in mammalian birth rates is fostered, through the administration of a composition comprising a component derived from the Indian Mulberry or Morinda citrifolia L. plant.

General Description of the Morinda citrifolia L. Plant

The Indian Mulberry or Morinda citrifolia plant is known scientifically as Morinda Citrifolia L. The plant is native to Southeast Asia and has spread in early times to a vast area from India to eastern Polynesia. It grows randomly in the wild, and it has been cultivated in plantations and small individual growing plots. Although the fruit has been eaten by several nationalities as food, the most common use of the Morinda citrifolia plant has traditionally been as a red and yellow dye source.

Processing Morinda citrifolia Leaves

The leaves of the Morinda citrifolia plant are one possible component of the Morinda citrifolia plant that may be present in some compositions of the present invention. For example, some compositions comprise leaf extract and/or leaf juice as described further herein. Some compositions comprise a leaf serum that is comprised of both leaf extract and fruit juice obtained from the Morinda citrifolia plant. Some compositions of the present invention comprise leaf serum and/or various leaf extracts as incorporated into a nutraceutical product (“nutraceutical” herein referring to any product designed to improve the health of living organisms such as human beings or mammals).

In some embodiments of the present invention, the Morinda citrifolia leaf extracts are obtained using the following process. First, relatively dry leaves from the Morinda citrifolia L. plant are collected, cut into small pieces, and placed into a crushing device—preferably a hydraulic press—where the leaf pieces are crushed. In some embodiments, the crushed leaf pieces are then percolated with an alcohol such as ethanol, methanol, ethyl acetate, or other alcohol-based derivatives using methods known in the art. Next, in some embodiments, the alcohol and all alcohol-soluble ingredients are extracted from the crushed leaf pieces, leaving a leaf extract that is then reduced with heat to remove all the liquid therefrom. The resulting dry leaf extract will herein be referred to as the “primary leaf extract.”

In some embodiments, the primary leaf extract is subsequently pasteurized. The primary leaf extract may be pasteurized preferably at a temperature ranging from 70 to 80 degrees Celsius and for a period of time sufficient to destroy any objectionable organisms without major chemical alteration of the extract. Pasteurization may also be accomplished according to various radiation techniques or methods.

In some embodiments of the present invention, the pasteurized primary leaf extract is placed into a centrifuge decanter where it is centrifuged to remove or separate any remaining leaf juice therein from other materials, including chlorophyll. Once the centrifuge cycle is completed, the leaf extract is in a relatively purified state. This purified leaf extract is then pasteurized again in a similar manner as discussed above to obtain a purified primary leaf extract.

Preferably, the primary leaf extract, whether pasteurized and/or purified, is further fractionated into two individual fractions: a dry hexane fraction, and an aqueous methanol fraction. This is accomplished preferably in a gas chromatograph containing silicon dioxide and CH2Cl2-MeOH ingredients using methods well known in the art. In some embodiments of the present invention, the methanol fraction is further fractionated to obtain secondary methanol fractions. In some embodiments, the hexane fraction is further fractionated to obtain secondary hexane fractions.

One or more of the leaf extracts, including the primary leaf extract, the hexane fraction, methanol fraction, or any of the secondary hexane or methanol fractions may be combined with the fruit juice of the fruit of the Morinda citrifolia plant to obtain a leaf serum (the process of obtaining the fruit juice to be described further herein). In some embodiments, the leaf serum is packaged and frozen ready for shipment; in others, it is further incorporated into a nutraceutical product as explained herein.

Processing Morinda citrifolia Fruit

Some embodiments of the present invention include a composition comprising fruit juice of the Morinda citrifolia plant. In some embodiments the fruit may be processed in order to make it palatable for human consumption and included in the compositions of the present invention. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another product, frozen or pasteurized. In some embodiments of the present invention, the juice and pulp can be pureed into a homogenous blend to be mixed with other ingredients. Other processes include freeze drying the fruit and juice. The fruit and juice can be reconstituted during production of the final juice product. Still other processes may include air drying the fruit and juices prior to being masticated.

In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, but preferably for 2 to 3 days. The fruit is ripened or aged by being placed on equipment so that the fruit does not contact the ground. The fruit is preferably covered with a cloth or netting material during aging, but the fruit can be aged without being covered. When ready for further processing the fruit is light in color, such as a light green, light yellow, white or translucent color. The fruit is inspected for spoilage or for excessive green color and firmness. Spoiled and hard green fruit is separated from the acceptable fruit.

The ripened and aged fruit is preferably placed in plastic lined containers for further processing and transport. The containers of aged fruit can be held from 0 to 30 days, but preferably the fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions. The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.). Another product manufactured is Morinda citrifolia puree and puree juice, in either concentrate or diluted form. Puree is essentially the pulp separated from the seeds and is different than the fruit juice product described herein.

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, a reverse osmosis filtration device, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The resulting pulp extract typically has a fiber content of 10 to 40 percent by weight. The resulting pulp extract is preferably pasteurized at a temperature of 181° F. (83° C.) minimum and then packed in drums for further processing or made into a high fiber product.

The filtered juice and the water from washing the wet pulp are preferably mixed together. The filtered juice may be vacuum evaporated to a brix of 40 to 70 and a moisture of 0.1 to 80 percent, more preferably from 25 to 75 percent. The resulting concentrated Morinda citrifolia juice may or may not be pasteurized. For example, the juice would not be pasteurized in circumstances where the sugar content or water activity was sufficiently low enough to prevent microbial growth.

Processing Morinda citrifolia Seeds

Some Morinda citrifolia compositions of the present invention include seeds from the Morinda citrifolia plant. In some embodiments of the present invention, Morinda citrifolia seeds are processed by pulverizing them into a seed powder in a laboratory mill. In some embodiments, the seed powder is left untreated. In some embodiments, the seed powder is further defatted by soaking and stirring the powder in hexane—preferably for 1 hour at room temperature (Drug:Hexane—Ratio 1:10). The residue, in some embodiments, is then filtered under vacuum, defatted again (preferably for 30 minutes under the same conditions), and filtered under vacuum again. The powder may be kept overnight in a fume hood in order to remove the residual hexane.

Still further, in some embodiments of the present invention, the defatted and/or untreated powder is extracted, preferably with ethanol 50% (m/m) for 24 hours at room temperature at a drug solvent ratio of 1:2.

Processing Morinda citrifolia Oil

Some embodiments of the present invention may comprise oil extracted from the Morinda Citrifolia plant. The method for extracting and processing the oil is described in U.S. patent application Ser. No. 09/384,785, filed on Aug. 27, 1999 and issued as U.S. Pat. No. 6,214,351 on Apr. 10, 2001, which is incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

### Compositions and Their Use

The present invention features compositions and methods for increasing mammalian birth rates comprising the administration of processed Morinda citrifolia based formulations. Embodiments of the present invention also comprise methods for internally introducing a Morinda citrifolia composition into the body of a mammal. Several embodiments of the Morinda citrifolia compositions comprise various different ingredients, each embodiment comprising one or more forms of a processed Morinda citrifolia component as taught and explained herein.

Compositions of the present invention may comprise any of a number of Morinda citrifolia components such as: extract from the leaves of Morinda citrifolia, leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, Morinda citrifolia seeds, Morinda citrifolia seed extracts, extracts taken from defatted Morinda citrifolia seeds, and evaporated concentration of Morinda citrifolia fruit juice. Compositions of the present invention may also include various other ingredients. Examples of other ingredients include, but are not limited to: artificial flavoring, other natural juices or juice concentrates such as a natural grape juice concentrate or a natural blueberry juice concentrate; carrier ingredients; and others as will be further explained herein.

Any compositions having the leaf extract from the Morinda citrifolia leaves, may comprise one or more of the following: the primary leaf extract, the hexane fraction, methanol fraction, the secondary hexane and methanol fractions, the leaf serum, or the nutraceutical leaf product.

In some embodiments of the present invention, active ingredients or compounds of Morinda citrifolia components may be extracted out using various procedures and processes commonly known in the art. For instance, the active ingredients may be isolated and extracted out using alcohol or alcohol-based solutions, such as methanol, ethanol, and ethyl acetate, and other alcohol-based derivatives using methods known in the art. These active ingredients or compounds may be isolated and further fractioned or separated from one another into their constituent parts. Preferably, the compounds are separated or fractioned to identify and isolate any active ingredients that might help to prevent disease, enhance health, or perform other similar functions. In addition, the compounds may be fractioned or separated into their constituent parts to identify and isolate any critical or dependent interactions that might provide the same health-benefiting functions just mentioned.

Any components and compositions of Morinda citrifolia may be further incorporated into a nutraceutical product (again, “nutraceutical” herein referring to any drug or product designed to improve the health of living organisms such as human beings or mammals). Examples of nutraceutical products may include, but are not limited to: intravenous products, topical dermal products, and various nutraceutical and other products as may be further discussed herein.

The compositions of the present invention may be formulated into any of a variety of embodiments, including oral compositions, topical dermal solutions, intravenous solutions, and other products or compositions.

Oral compositions may take the form of, for example, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia components in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain the Morinda citrifolia components in admixture with excipients suitable for the manufacture of aqueous suspensions.

Typical sweetening agents may include, but are not limited to: natural sugars derived from corn, sugar beets, sugar cane, potatoes, tapioca, or other starch-containing sources that can be chemically or enzymatically converted to crystalline chunks, powders, and/or syrups. Also, sweeteners can comprise artificial or high-intensity sweeteners, some of which may include aspartame, sucralose, stevia, saccharin, etc. The concentration of sweeteners may be between from 0 to 50 percent by weight of the Morinda citrifolia composition, and more preferably between about 1 and 5 percent by weight.

Typical flavoring agents can include, but are not limited to, artificial and/or natural flavoring ingredients that contribute to palatability. The concentration of flavors may range, for example, from 0 to 15 percent by weight of the Morinda citrifolia composition. Coloring agents may include food-grade artificial or natural coloring agents having a concentration ranging from 0 to 10 percent by weight of the Morinda citrifolia composition.

Typical nutritional ingredients may include vitamins, minerals, trace elements, herbs, botanical extracts, bioactive chemicals, and compounds at concentrations from 0 to 10 percent by weight of the Morinda citrifolia composition. Examples of vitamins include, but are not limited to, vitamins A, B1 through B12, C, D, E, Folic Acid, Pantothenic Acid, Biotin, etc. Examples of minerals and trace elements include, but are not limited to, calcium, chromium, copper, cobalt, boron, magnesium, iron, selenium, manganese, molybdenum, potassium, iodine, zinc, phosphorus, etc. Herbs and botanical extracts may include, but are not limited to, alfalfa grass, bee pollen, chlorella powder, Dong Quai powder, Echinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

The ingredients to be utilized in a topical dermal product may include any that are safe for internalizing into the body of a mammal and may exist in various forms, such as gels, lotions, creams, ointments, etc., each comprising one or more carrier agents. The ingredients or carrier agents incorporated into systemically (e.g., intravenously) administered compositions may also comprise any known in the art.

In one exemplary embodiment, a Morinda citrifolia composition of the present invention comprises one or more of a processed Morinda citrifolia component present in an amount by weight between about 0.01 and 100 percent by weight, and preferably between 0.01 and 95 percent by weight. Several embodiments of formulations are included in U.S. Pat. No. 6,214,351, issued on Apr. 10, 2001, which are herein incorporated by reference. However, these compositions are only intended to be exemplary, as one ordinarily skilled in the art will recognize other formulations or compositions comprising the processed Morinda citrifolia product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

The processed Morinda citrifolia product is the active ingredient or contains one or more active ingredients, such as quercetin, rutin, scopoletin, octoanoic acid, potassium, vitamin C, terpenoids, alkaloids, anthraquinones (such as nordamnacanthal, morindone, rubiandin, B-sitosterol, carotene, vitamin A, flavone glycosides, linoleic acid, Alizarin, amino acides, acubin, L-asperuloside, caproic acid, caprylic acid, ursolic acid, and a putative proxeronine and others, for productively affecting mammalian reproductive systems. Active ingredients may be extracted utilizing aqueous or organic solvents including various alcohol or alcohol-based solutions, such as methanol, ethanol, and ethyl acetate, and other alcohol-based derivatives using any known process in the art. The active ingredients of quercetin and rutin are present in amounts by weight ranging from 0.01-10 percent of the total formulation or composition. These amounts may be concentrated as well into a more potent concentration in which they are present in amounts ranging from 10 to 100 percent.

### EXAMPLES

The following example illustrates some of the embodiments of the present invention comprising the administration of a composition comprising components of the Indian Mulberry or Morinda citrifolia L. plant. These examples are not intended to be limiting in any way, but are merely illustrative of benefits, advantages, and remedial effects of some embodiments of the Morinda citrifolia compositions of the present invention.

As illustrated by the following Example, embodiments of the present invention have been tested. Specifically, the Example illustrates the results of in-vitro studies that confirmed that concentrates of processed Morinda citrifolia products (“TNJ” is an evaporative concentrate) could have productive affects on mammalian reproductive systems. The percentage of concentration refers to the concentration strength of the particular concentrate tested; that is, the strength of concentration relative to the processed Morinda citrifolia product from which the concentrate was obtained.

### Example 1

Three separate experiments were conducted to determine whether administration of TNJ could increase the birth rate in ICR mice. The following are results from the three experiments conducted.

In experiment 1 and 2, ten male and twenty five female eight-week old ICR mice were utilized. One male and two female were mated in the same cage. The day that a vaginal plug was detected was established as day 0 of recorded gestation. Twenty pregnant mice were divided into two groups: 5% TNJ was supplied to 10 pregnant mothers beginning at day 0 while the control group was supplied with regular drinking water. All pregnant mice were sacrificed by cervical dislocation at gestation day 18. At day 18, the fetuses were removed from the uterus, the number of fetus per each mother was recorded and each fetus was examined for malformation. There were no malformed fetuses found in TNJ group, while 1% malformed fetuses were found in control group. Data from the experiments is shown below in Tables 1 and 2.

TABLE 1
Numbers of fetus in control and 5% TNJ group

| | Control | 5% TNJ |
|---|---|---|
| Mean ± SD | 11.2 ± 1.1 | 14.5 ± 2.9 |
| Mother # | 10 | 10 |
| P value | | <0.05 |
| Malformed rate | 1% | 0.00 |

TABLE 2
Numbers of fetus in control and 5% TNJ group

| | Control | 5% TNJ |
|---|---|---|
| Mean ± SD | 11.9 ± 2.0 | 15.5 ± 3.6 |
| Mother # | 10 | 10 |
| P value | | <0.01 |
| Malformed rate | 1% | 0.00 |

In experiment 3, ten male mice from experiment 2 were divided into two groups: 5% TNJ was supplied to five adult male ICR mice for one week, another five were supplied with regular drinking water as a control. An additional twenty-five female mice were mated with male either on the TNJ or water at 1:2 ratio of male and female in one cage. The pregnancy rate of the control group was 85%, while the pregnancy rate of the TNJ group was 100% at three days. Gestation day 0 was recorded when a positive vaginal plug was detected. 5% TNJ was supplied to 13 pregnant mothers from gestation day zero through pregnancy, birth and until the offspring reached 21 days of age. All fetuses were separated from their mother at the 21st day after delivery. The babies born to mothers consuming TNJ were put on the regular drinking water after the 21^{st} day. The fetal number of each mother in control and Noni group were examined on daily basis to observe the illness, death, and health conditions.

Both groups were examined for malformed fetuses. No malformed fetuses were found in the TNJ group, while a 1% malformation rate was observed in the control group. The pregnancy rate was 100% after three days when the female mated with male pretreated with TNJ for one week, while the pregnancy rate of the control group remained 85%. Accordingly, TNJ may improve sperm quality and/or promote the production of the sperms in male pretreated with TNJ for one week.

TABLE 3
Numbers of fetus in control and 5% TNJ groups

| Number of Fetus | Control | 5% TNJ |
|---|---|---|
| Mean ± SD | 9.9 ± 2.0 | 13.2 ± 3.0 |
| Mother # | 10 | 13 |
| P value | | <0.01 |
| Malformed rate | 1% | 0.00 |

In Summary, TNJ increases the birth rate in pregnant ICR mice. Accordingly, administration of products containing TNJ may be utilized to provide benefit to individuals and/or animals which desire higher birth rates.

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

What is claimed is:

1. A formulation adapted to increase mammalian birth rates comprising:
processed Morinda citrifolia fruit juice.

2. The formulation of claim 1, further comprising a Morinda citrifolia product selected from a group consisting of: extract from the leaves of Morinda citrifolia, leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

3. The formulation of claim 1, further comprising an element selected from a list consisting of grape juice, blueberry juice and apple juice.

4. The formulation of claim 1, wherein said:
processed Morinda citrifolia fruit juice is present in an amount by weight between about 85-99.99 percent.

5. The formulation of claim 1, further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

6. The formulation of claim 1, wherein said formulation further comprises an active ingredient Quercetin present in an amount between about 0.01 and 10 percent by weight.

7. The formulation of claim 1, wherein said formulation further comprises an active ingredient Rutin present in an amount between about 0.01 and 10 percent by weight.

8. The formulation of claim 1, wherein said formulation is formulated for administering in a way selected from a list consisting of orally, topically, and intravenously.

9. A method of treating low birth rates in a mammal, which comprises:
processing a Morinda citrifolia product;
administering to said mammal a formulation comprising an effective amount of a processed Morinda citrifolia product; and
increasing the birth rate of said mammal.

10. The method of claim 9, wherein said processed Morinda citrifolia product comprises a processed Morinda citrifolia selected from a group consisting of: extract from the leaves of Morinda citrifolia, leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

11. The method of claim 9, wherein the formulation further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

12. The method of claim 9, wherein the processing step comprises the steps of: adding a Morinda citrifolia product a solvent; and isolating an active ingredient from said Morinda citrifolia product.

13. The method of claim 12, wherein the solvent is selected from a list consisting of water, ethanol, butanol, isoproponal and ethyl acetate.

14. A method for treating a mammal comprising the steps of:
obtaining a processed Morinda citrifolia freeze dried extract comprising the steps of:
freezing one or more Morinda citrifolia products;
defrosting said Morinda citrifolia product;
chopping said Morinda citrifolia product into small pieces;
adding an identified amount of distilled water to said Morinda citrifolia product to obtain a solution;
agitating said solution at an identified temperature for an identified period of time;
freeze-drying said supernatant solution to obtain said processed Morinda citrifolia product extract;
preparing a formulation comprising said processed Morinda citrifolia extract;
administering said nutraceutical to a patient; and
increasing the birth rate of said patient.

15. The method of claim 14, further comprising the steps of:
extracting said solution with a solvent for an identified period of time;
removing any solids in said solution;
extracting the solvent from said solution under decreasing pressure; and
filtering any solids produced to obtain a supernatant solution after adding water but before agitating the solution.

16. The method of claim 14, wherein the solvent is selected from a list consisting of ethanol, methanol, butanol and ethyl acetate.

17. The method of claim 14, wherein said formulation further comprises a processed Morinda citrifolia hot water extract.

18. The method of claim 14, wherein said formulation further comprises a processed Morinda citrifolia steam distilled extract.

19. The method of claim 14, wherein said formulation further comprises processed Morinda citrifolia product selected from a list consisting of fruit juice, puree juice and dietary fiber.
